(19) United States
(12) Patent Application Publication
(10) Pub. No.: US 2021/0186812 A1
(43) Pub. Date: Jun. 24, 2021

(54) System for Recovering Autologous Thrombin

(71) Applicant: Patrick Pennie, Fort Myers, FL (US)

(72) Inventor: Patrick Pennie, Fort Myers, FL (US)

(21) Appl. No.: 17/122,129

(22) Filed: Dec. 15, 2020

Related U.S. Application Data
(60) Provisional application No. 62/949,832, filed on Dec. 18, 2019.

(51) Int. Cl. A61J 1/20; A61J 1/14; A61M 5/178

(52) U.S. Cl. CPC A61J 1/2086; A61J 1/1456; A61M 5/1782; A61M 2202/0425; A61M 2205/7527; A61M 2202/0427; A61M 2202/0445; A61M 2202/0429; A61M 2202/0415

(57) ABSTRACT

A system is disclosed for recovering autologous thrombin from a blood product. The blood product is drawn into an injecting syringe and a clot is allowed to form. A discharge port of the injecting syringe is communicably connected to an intake port of an aspirating syringe through a hydrophilic filter assembly. The injecting and aspirating syringes are simultaneously operated to transmit blood product through the filter such that fibrous clotting material and red blood cells are removed by the filter and a concentrated, high-quality thrombin solution is recovered.

### RELATED APPLICATION

This application claims the benefit of Provisional Application Ser. No. 62/949,832 filed Dec. 18, 2019.

### FIELD OF THE INVENTION

This invention relates to an improved apparatus and method for recovering an autologous thrombin solution from a sample of an individual's blood, which can then be used to promote clotting and activate PRP growth factors in conjunction with dental and orthopedic surgical procedures.

### BACKGROUND OF THE INVENTION

Thrombin is a blood enzyme that is useful for activating procoagulant and platelet rich plasma (PRP) growth factors needed during dental and orthopedic surgeries. Thrombin is present in both human and animal blood. Traditionally, bovine thrombin has been utilized for clotting and activating the growth factors of PRP employed in surgical procedures. However, the results of using enzymes derived from this source have been less than optimal. Moreover, mad cow disease has presented a serious health risk that has made the use of bovine derived thrombin much less desirable.

Recently, the use of autologous thrombin derived from the patient's own blood has become the preferred manner of obtaining the thrombin enzyme. Typically, a sample of the patient's blood is drawn and allowed to clot in a petri dish or other appropriate container. The blood product is centrifuged to separate a yellow thrombin solution from the clot. The thrombin solution or supernatent is then aspirated and calcium, in the form of calcium chloride, is added to the solution to yield the final blood clotting thrombin product.

The foregoing process exhibits a number of disadvantages. It is both unduly time consuming and involves a number of discrete steps that must be precisely performed. A centrifuge and a source of calcium are both required. Moreover, the thrombin supernatant that is produced is often of a poor quality and does not provide for the desired clotting and growth activation factors. As a result, the results obtained by using such autologous thrombin in dental and surgical procedures is frequently less than optimal.

### SUMMARY OF THE INVENTION

It is therefore an object of the present invention to provide an apparatus and method for recovering a high-quality autologous thrombin solution from an individual's blood, which overcomes the problems exhibited by prior art techniques.

It is a further object of this invention to provide an apparatus and method for recovering an improved high-quality autologous thrombin that activates procoagulant factors and PRP growth factors much more effectively than has been achieved using previously produced thrombin solutions.

It is a further object of this invention to provide a much simpler and more efficient system for producing autologous thrombin that reduces the time, complexity, equipment, supplies and steps conventionally needed to produce autologous thrombin for surgical procedures.

It is a further object of this invention to provide a system for recovering a very high quality and effective autologous thrombin for use in dental and orthopedic surgical procedures.

It is a further object of this invention to provide a system for recovering a high-quality autologous thrombin that effectively clots to reduce excessive bleeding during surgery and which activates PRP growth factors to more effectively seal and repair bones and stimulate bone growth and recovery.

It is a further object of this invention to provide a system for recovering autologous thrombin that is safer and more effective for use in surgery than conventional bovine thrombin solutions and more efficient and effective than previous autologous thrombin products.

It is a further object of this invention to provide a method for recovering a high-quality autologous thrombin solution, which exhibits a higher purity and eliminates much of the fibrous clotting materials and blood cells that are present in conventional autologous thrombin solutions.

This invention features an autologous thrombin recovery system including injecting and aspirating syringes and a filter assembly operably interconnected between the injecting and aspirating syringes. The injecting syringe includes a cylindrical supply receptacle for holding a blood product and having a discharge port formed therein. A first piston assembly is operatively interconnected with the supply receptacle of the injecting syringe. The aspirating syringe includes a receiving receptacle having a fluid inlet port formed therein and a second aspirating piston assembly operatively interconnected thereto. The filter assembly includes a fluid intake port for communicably connecting to the discharge port of the injecting receptacle and a fluid outlet port for communicably connecting to the inlet port of the aspirating syringe. A filter component is disposed between the intake and outlet ports of the filter assembly. In operation, the first piston assembly of the injecting syringe is manually driven through the supply receptacle of the injecting syringe and the aspirating syringe is simultaneously operated by withdrawing or retracting the second piston assembly through the receiving receptacle of the aspirating syringe. This causes blood product to be transmitted through the filter component of the filter assembly such that clotted and fibrous material, as well as red blood cells are removed from the blood product by the filter component. A solution of platelet rich plasma (PRP) and thrombin are thereby collected in the receiving receptacle of the aspirating syringe. This collected thrombin solution may then be used for various dental and orthopedic surgical applications.

In a preferred embodiment, the discharge port of the injecting syringe and the inlet port of the aspirating syringe include male luer ports. The intake port of the filter assembly may include a female luer port. The outlet port of the filter assembly may include a male luer port. A connector fitting having a pair of female luer port connections may be used to connect the male outlet port of the filter assembly to the male inlet port of the aspirating syringe. One of the female luer port connections of the connector fitting may be attached to the outlet port of the filter assembly and the other female luer port connector may be communicably secured to the inlet port of the aspirating syringe. The female connections of the connector fitting are themselves communicably interconnected. As a result, when the first piston assembly of the injecting syringe is depressed through the supply receptacle and the second piston assembly of the aspirating syringe is simultaneously retracted, blood product is dispensed through the male discharge port of the injecting syringe and transmitted through the interconnected intake port of the filter assembly. The filter component separates the coagulated, fibrous and cellular matter from the blood product. A high-quality thrombin and plasma solution is then transmitted through the outlet port of the filter assembly, through the connector fitting and through the attached inlet port of the aspirating syringe into the receiving receptacle of that syringe.

The injecting syringe preferably includes a glass supply receptacle, which promotes coagulation of the blood. After blood product is introduced into the supply receptacle, it may be agitated for a predetermined time to further facilitate coagulation prior to operation of the system.

The filter component may include a hydrophilic filter. The filter preferably includes a pore size of approximately 0.2 microns. In any event, the filter component should include-pores having a size not larger than 0.45 microns apiece.

### BRIEF DESCRIPTION OF THE DRAWINGS

Other objects, features and advantages will occur from the following description of a preferred embodiment and the accompanying drawings, in which:

FIG. 1 is an elevational schematic view of the system for recovering autologous thrombin in accordance with this invention and wherein a blood product is contained in the injecting syringe of the system before the filtering operation is performed; and

FIG. 2 is a view similar to FIG. 1 of the system after the filtering operation step has been performed and with a purified and high-quality plasma/PRP thrombin solution contained in the aspirating syringe.

### DETAILED DESCRIPTION OF PREFERRED EMBODIMENTS

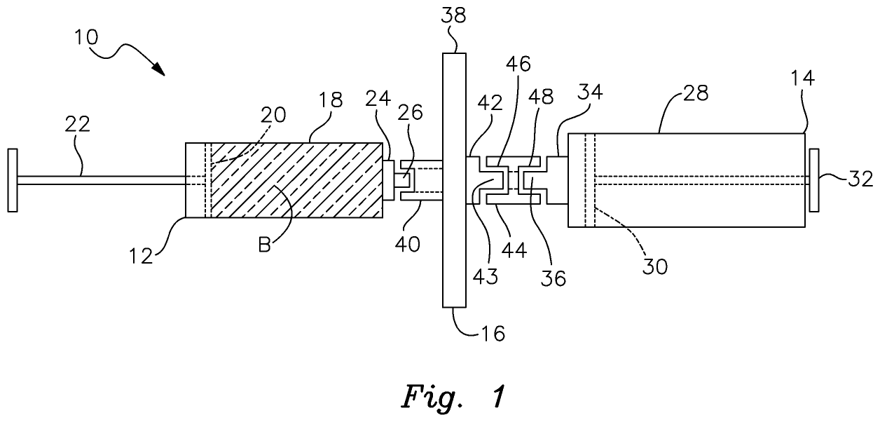

There is shown in FIG. 1 an apparatus 10 for recovering autologous thrombin in accordance with this invention. In particular the apparatus and method of this invention are intended for retrieving an improved, high-quality thrombin solution (comprising the enzyme thrombin in solution with plasma and platelet rich plasma). The thrombin activates the clotting or anticoagulation factors of the blood, as well as the PRP growth factors present in the solution. As a result, the solution is extremely effective for use in a variety of dental and orthopedic surgical procedures where bone repair and growth are desired. The particular type of surgical procedure for which the thrombin solution may be used is not a limitation of this invention.

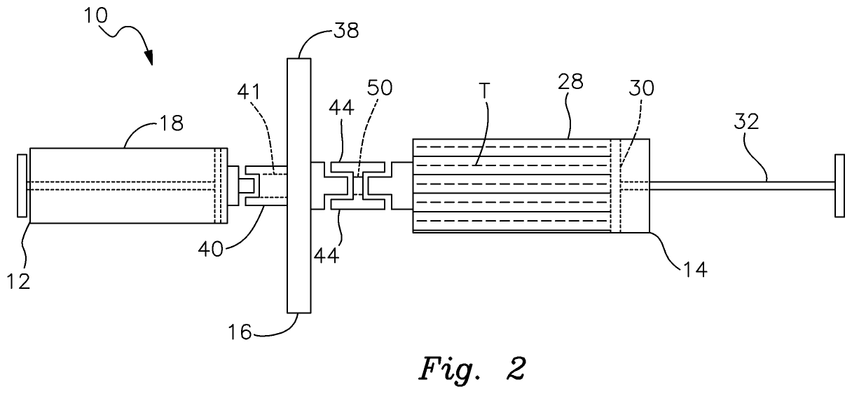

Autologous thrombin solution is recovered from a patient undergoing a dental or orthopedic surgical procedure using a system comprising apparatus 10 depicted schematically in FIGS. 1 and 2 operated in accordance with the method of this invention. In particular, apparatus 10 includes a pair of hypodermic syringes 12 and 14 that are operably and communicably interconnected by a filter assembly 16. Syringes 12 and 14 preferably comprise sterile medical grade syringes suitable for use in processing blood and other biological liquids. Syringe 12 constitutes an injecting syringe and includes a generally cylindrical supply receptacle 18, which is preferably composed of glass. As is described more fully below, the glass receptacle promotes clotting of the blood product B within receptacle 18 when the method of this invention is practiced. Injecting syringe 12 also includes a standard first piston assembly featuring a diaphragm, disc or piston 20 that is slidably and sealably mounted within receptacle 18 such that the peripheral surface of the piston sealably interengages the inner wall of receptacle 18 and is slidable back and forth through the receptacle. Piston 20 is driven through receptacle 18 by a manually operated plunger 22 that is mounted within the receptacle 18 of syringe 12 in a conventional manner.

Injecting syringe 12 also includes a discharge port 24, which preferably features a male luer port having a threaded connective tip 26 of the type that will be known to persons skilled in the art. Discharge port 24 is formed at the lower or distal end of supply receptacle 18 and communicably interconnected with an interior chamber of the receptacle. Tip 26 allows syringe 12 to be operatively interconnected to filter 16 in the manner described below.

Second syringe 14 comprises a receiving or aspirating syringe that may have a configuration or construction identical or similar to that of injecting syringe 12. In particular, syringe 14 includes a cylindrical fluid-receiving receptacle 28. Each of syringe receptacles 18 and 28 may have various capacities within the scope of this invention. Syringe 14 operatively supports a second piston assembly comprising a diaphragm, disc or piston 30 that is slidably and sealably mounted within the interior chamber of receptacle 28. A plunger 32 is operatively mounted to piston 30 and extends outwardly from the upper or proximal end of receptacle 28. Plunger 32 is pulled rearwardly or retractably to draw piston 30 through receptacle 28 so that aspiration is performed in the manner described more fully below.

Aspirating syringe 14 likewise includes a male luer port 34 that communicates with the interior chamber of receptacle 28 and is carried at the lower or distal end of that receptacle. Luer port 34 includes a threaded tip 36 that, again, allows syringe 14 to be operatively interconnected to filter assembly 16 in the manner described below. Port 34 thereby serves as an inlet port of aspirating syringe 14.

Syringes 12 and 14 are operatively interconnected by filter assembly 16. More specifically, the filter assembly includes a hydrophilic filter 38 featuring a pore size that is not larger than 0.45 microns. A particularly preferred pore size is 0.2 microns for most effectively performing the thrombin recovery process of this invention.

A filter intake port 40 is communicably connected to a first side of hydrophyllic filter 38 and a filter outlet port 42 is likewise communicably connected to the opposite side of filter 38. Filter intake port 40 preferably comprises a female luer-type connective port that is threadably or otherwise operatively interconnected to male discharge port 24 of injecting syringe 12. Filter outlet port 42 likewise preferably comprises a male luer port. An internal passageway 41 is formed in intake port 40 of filter assembly 16, which permits blood product to be transmitted from syringe 12 to filter component 38 as described more fully below. Filter assemblies featuring intake and outlet ports and an intervening filter, as specified herein, are conventionally available to persons skilled in the art. Such filter assemblies are used in IV circuits, for example, to vent and filter air and restrict contamination in an IV line. Alternatively, constructing such assemblies will be understood by such persons.

Male outlet 42 of filter assembly 16 is operatively and communicably interconnected to male inlet port 34 of syringe 14 by means of a coupler or connector fitting 44 having a pair of opposing and communicably interconnected female luer ports 46 and 48. Each port 46, 48 includes interior threads that are connected to corresponding threads on tips 43 and 36 of male luer ports 42 and 34 respectively. This allows the aspirating syringe 14 to be securely and communicably interconnected to filter assembly 16 through connector fitting 44. A central passageway 50 formed through fitting 44 allows filtered supernatant solution to be transmitted through the connector fitting between filter outlet 42 and syringe inlet port 34. Without connector fitting 44 featuring a pair of opposing and communicating female ports, the aspirating syringe would be unable to operatively interconnect with a filter assembly of the type represented by assembly 16. Preferably, respective receptacles 18 and 28 of syringes 12, 14 are permanently enclosed and liquids are introduced and discharged from the syringes by conventional injection and aspiration operations performed using the first and second piston assemblies respectively. Alternatively, it should be noted that, each syringe 12, 14 may include a cap or closure that is selectively opened and closed, as needed, to introduce and remove blood products and filtered thrombin solution to and from the syringes respectively when the system is used in accordance with this invention.

System 10 is employed to recover autologous thrombin from a patient in accordance with the following procedure. Initially, a sample of the patient's blood is drawn into receptacle 18 of syringe 12 and the syringe is capped. The blood can be drawn into the syringe in a standard manner or in alternative ways that are sterile and medically acceptable. The blood product B (FIG. 1) is then agitated for a selected duration to promote clotting and typically until a clot forms within receptacle 18. After clotting occurs in blood product B, syringe 12 is operatively interconnected to intake port 40 of filter assembly 16. Specifically, exteriorly threaded tip 26 of male luer discharge port 24 is threadably interconnected interiorly threaded with female luer port 40 of filter assembly 16.

Receiving or aspirating syringe 14 is likewise operatively connected to outlet port 42 of filter assembly 16. In particular, the exteriorly threaded tip 36 of male inlet port 34 is interengaged with the interiorly threaded female port 48 of coupler 44. By the same token, interiorly threaded female port 46 of coupler fitting 44 is interengaged with exteriorly threaded tip 43 of filter outlet 42.

After the foregoing luer port interconnections are completed, syringes 12 and 14 are operated to transmit blood product B from receptacle 18 of syringe 12 through filter assembly 16 and into receptacle 28 of syringe 14. Specifically, plunger 22 is actuated or depressed from the position shown in FIG. 1 to that shown in FIG. 2. This drives piston 20 through the interior chamber of supply receptacle 18 of syringe 12. The clotted blood product is thereby injected through discharge port 24 of syringe 12 and passageway 41 of interconnected filter intake port 40. Plunger 32 of syringe 14 is simultaneously retracted or withdrawn through receptacle 28 from the position shown in FIG. 1 to the position shown in FIG. 2. This simultaneous dual operation of syringes 12 and 18 pulls the blood product forcefully through filter assembly 16. Hydrophilic filter 38 traps the clot and effectively removes unwanted constituents such as fibrous material, blood proteins and red blood cells from the blood product. More specifically, the simultaneous dual syringe operation provides a sufficient level of pressure to more easily, smoothly and effectively transmit the blood product through the relatively small pore-size hydrophilic filter, which largely eliminates the clot, fibrous material and red blood cells from the blood product. As a result, a thrombin solution comprising plasma that contains high quality PRP and thrombin enzyme is transmitted through filter outlet 42, as well as interconnected coupler fitting 44 and inlet port 34 of syringe 14. As shown in FIG. 2, filtered thrombin solution T, comprising plasma, PRP and high-quality thrombin is collected within receptacle 28 of syringe 14.

After filtration is completed, receiving syringe 14 is disengaged from coupler fitting 44. A concentrated autologous thrombin solution or supernatant remains in the receptacle of the receiving syringe. This supernatant is then retrieved from syringe 16 and mixed with either whole blood or PRP. The thrombin activates clotting and growth factors in the PRP that allow the mixture to be used effectively in a variety of dental and orthopedic surgical applications, including bone sealing, reinforcement, repair and reconstruction. The use of the patient's own PRP and thrombin achieves results that are far superior to those obtained using thrombin and PRP that are nonautogenous.

System 10 and the method of recovering autologous thrombin using that system provide for a number of advantages over the prior art. The disclosed system produces a thrombin solution that is far safer and more effective for use in dental and orthopedic surgical applications than are bovine thrombin products. Medical risks associated with mad cow disease are eliminated. The present invention enables very high-quality autologous thrombin solution to be obtained much faster, more efficiently and using less equipment, time and materials than has previously been involved. Centrifuging the blood product and related centrifuge equipment are not needed. Likewise, this system does not require the addition of calcium to compensate for the anticoagulation effects that normally result when a citrate solution is added to a PRP blood source.

By using a glass delivery syringe, the disclosed system also eliminates the use of glass powder for promoting clotting during the autologous thrombin recovery process. The highly concentrated and high-quality thrombin recovered using the apparatus and process of this invention provides for significantly improved surgical and orthopedic results. Bone regeneration, support and recovery are aided considerably. PRP growth factors and clotting are likewise significantly improved by the concentrated thrombin solution. This enables much better bone growth, healing, regeneration and support to be achieved. Various types of dental and orthopedic surgical procedures are thereby benefitted.

Accordingly, the present invention relates to a system and method for recovering autologous thrombin by using a hydrophilic filter that is communicably interconnected between a pair of injecting and aspirating syringes. The system is operated to effectively remove fibrous clotting material and red blood cells from a blood sample such that a concentrated and high-quality thrombin solution is recovered for use in PRP related dental and orthopedic surgical applications.

Although specific features of the invention are shown in some of the drawings and not others, this is for convenience only, as each feature may be combined with any and ail of the other features in accordance with this invention.

What is claimed is:

1. An autologous thrombin recovery system comprising:
an injecting syringe including a supply receptacle for holding a blood product and having a discharge port formed therein, said injecting syringe further including a first piston assembly operatively interengaged with said supply receptacle;
an aspirating syringe including a receiving receptacle having a fluid inlet port formed therein and a second piston assembly operatively engaged with said receiving receptacle; and
a filter assembly operably interconnected between said injecting and aspirating syringes, said filter assembly including a fluid intake port for communicably connecting to said discharge port of said injecting receptacle and a fluid outlet port for communicably connecting to said inlet port of said aspirating syringe, said filter assembly further including a filter component disposed between and communicably interconnecting said intake and outlet ports of said filter assembly; said first piston assembly of said injecting syringe being driven through said supply receptacle of said injecting syringe and said second piston assembly being simultaneously retracted through said receiving receptacle of said aspirating syringe for transmitting the blood product from said supply receptacle of said injecting syringe through said filter component of said filter assembly such that constituent components of the blood product are removed by said filter component and a solution containing thrombin is collected in said receiving receptacle of said aspirating syringe.

2. The system of claim 1 in which said discharge port of said injecting syringe and said inlet port of said aspirating syringe include male luer ports.

3. The system of claim 2 in which said intake port of said filter assembly includes a female luer port.

4. The system of claim 3 in which said outlet port of said filter assembly includes a male luer port.

5. The system of claim 4 further including a connector fitting having a pair of female luer ports for communicably interconnecting to said male outlet port of said filter assembly and said male inlet port of said aspirating syringe respectively.

6. The system of claim 1 in which said injecting syringe includes a glass supply receptacle to promote coagulation of the blood.

7. The system of claim 1 in which said filter component includes a hydrophilic filter.

8. The system of claim 1 in which said filter component includes a pore size of approximately 0.2 microns.

9. The system of claim 1 in which said filter component includes pores having a size not greater than 0.45 microns apiece.

10. The system of claim 1 in which said filter component is adapted for removing at least one of the constituent blood components consisting of clotted material, fibrous material, one or more blood proteins and red blood cells.

11. The system of claim 1 in which the constituent components removed by said filter component include at least one of clotted material, fibrous material, one or more blood proteins, and red blood cells.

12. The system of claim 1 in which said thrombin solution includes plasma, platelet rich plasma and thrombin enzyme.

13. A method for recovering autologous thrombin from a blood product comprising the steps of:
introducing a blood product into the receptacle of an injecting syringe;
permitting a clot to form in the blood product;
communicably interconnecting a discharge port of the injecting syringe to an inlet port of an aspirating syringe through a filter assembly; and
simultaneously operating the injection and aspirating syringes to transmit the blood product through the filter assembly and remove constituent components of the blood product such that a thrombin solution is delivered to the receiving receptacle of the aspirating syringe.

14. The method of claim 13 in which the constituent components removed from the blood product include at least one of clotted material, fibrous material, one or more blood proteins, and red blood cells.

15. The method of claim 13 further including the step of agitating the blood product in the receptacle of the injecting syringe to promote formation of the clot in the blood product.

16. The method of claim 13 in which the blood product is transmitted through a hydrophilic filter of the filter assembly.
